(19) United States
(12) Patent Application Publication
(10) Pub. No.: US 2005/0101879 A1
(43) Pub. Date: May 12, 2005

(54) Needle aspiration biopsy device and method

(76) Inventors: Vinod B. Shidham, Elm Grove, WI (US); Anjani Shidham, Elm Grove, WI (US)

Correspondence Address: QUARLES & BRADY LLP, 411 E. WISCONSIN AVENUE, SUITE 2040, MILWAUKEE, WI, 53202-4497, US

(21) Appl. No.: 10/773,691

(22) Filed: Feb. 6, 2004

Related U.S. Application Data
(60) Provisional application No. 60/518,069, filed on Nov. 6, 2003.

(51) Int. Cl. A61B 10/00

(52) U.S. Cl. 600/566

(57) ABSTRACT

A needle aspiration biopsy device includes a syringe, a valve coupler for controlling a vacuum in the syringe barrel, and an anti-reflux head. The head includes a hub, defining a large collection well preferably several hundred micro liters in volume, and a needle mounted to the hub. A passageway is defined either by a contoured needle or a straight needle and an internal channel in the hub wall such that a proximal end of the passageway extends inside the hub and opens in spaced relation to a floor of the collection well. A method of biopsy using the device is also disclosed.

### CROSS-REFERENCE TO RELATED APPLICATIONS

This application claims benefit to U.S. provisional patent application Ser. No. 60/518,069 filed on Nov. 6, 2003.

### STATEMENT OF FEDERALLY SPONSORED RESEARCH OR DEVELOPMENT

Not applicable.

### BACKGROUND OF THE INVENTION

1. Field of the Invention

The present invention relates to medical devices, and in particular to needle aspiration biopsy devices.

2. Background of the Art

Fine needle aspiration biopsy (hereinafter “FNAB”) is a widely practiced technique for acquiring diagnostic sample specimen of various tumors and lesions, and is generally considered a safe, rapid and economical non-surgical alternative to conventional surgical biopsy.

The FNAB technique is conventionally practiced with a standard hypodermic needle syringe with a fine gauge needle, possibly suited with special syringe grips and modified to include a small specimen collection chamber at the base of the needle. The FNAB technique as conventionally practiced generally consists of inserting the needle of the syringe into the tumor, lesion or tissue of interest and then pulling the syringe piston to draw a vacuum in the needle before or while the tissue is probed several times in a to and fro motion. The vacuum and reciprocating motion of the syringe aspirates the tissue and causes specimens to pass through the needle and into the small space at the top of the needle. After some specimens have been collected, the piston is returned to the bottom of the syringe barrel to break the vacuum in the needle and the needle is withdrawn from the tissue. The needle is then removed from the syringe and the collected specimens are squirted onto a glass slide for analysis.

Conventional FNAB techniques can often yield insufficient specimens to conduct a thorough diagnostic analysis. This problem arises because of several deficiencies in conventional FNAB devices, particularly with regard to the delivery of the specimens into the needle, their retention in the device, and their transfer from the device to the diagnostic stage or vessel. Among other things, these deficiencies are in part due to the insufficient space for specimens, the way the specimens are introduced into the device and the way the vacuum is achieved and released.

More specifically, conventional hypodermic needles used for FNAB produces typically have a collection space of about 10-50 micro liters (μL). A 50 μL sample is usually enough specimens to conduct a single study, much less than this amount would likely not provide for meaningful analysis. The small collection space means that less specimens can be collected per biopsy. It also means that to maximize yield, the available space must essentially be filled with specimens in order to obtain a sufficient quantity for meaningful analysis.

In conventional FNAB devices specimens are susceptible to being drawn into the syringe barrel under vacuum or lost be reflux back through the needle, in both cases diminishing the overall yield usable for analysis. This problem is exacerbated by coagulation of the aspirated specimens in the collection chamber. Another problem with the small collection space is that small micro-specimen or portions of the accumulated specimens cannot be readily transferred to the examination site in a controlled manner. The narrowness of the opening essentially requires the specimens to be dumped or poured out into the test tube or onto the slide, and thus the more accurate “pick and spread” transfer method may not be used because conventional forceps may be too large.

U.S. Pat. No. 5,645,537 to Powles et al. discloses a needle aspiration biopsy device in which a body to which a syringe and needle are attached defines a relatively large internal collection chamber. The body is preferably formed of two parts with a joint formed across the collection chamber so that following the biopsy procedure a top of the body can be removed to allow access to the collection chamber. This device is disadvantageous, however, because collected specimens are lost by reflux back through the needle.

As illustrated by the above patent, conventional FNAB devices are susceptible to losing part of the sample through reflux of the collected specimens back through the needle. This arises in part because the vacuum in the needle is at all times in communication with the vacuum in the syringe barrel. To prevent the accumulated specimens in the collection well from entering the syringe barrel when the needle is withdrawn from the tissue, the vacuum must be broken before the needle is removed. The vacuum is released by returning the syringe piston to the initial retracted position in the barrel. This invariably causes reflux of the specimens back into the tissue by virtue of evacuated air being pushed from the syringe barrel through the collection chamber and out the needle. The typical way to make up for the specimens lost by reflux is to conduct multiple biopsies and/or increase the number of probes into the tissue per biopsy, thus increasing the time of the procedure and the patient's discomfort.

U.S. Pat. No. 4,549,554 to Markham and U.S. Pat. No. 5,413,115 to Baldwin disclose FNAB devices in which valves are interposed between the needles and syringes. The valves are designed to control the vacuum in the needle, and thereby avoid the aforementioned problem contributing to reflux. Both of these devices include a simple open-shut valve having an opening that can be slid or rotated into and out of alignment with the passageway between the needle and the syringe and thereby open and close off, respectively, the vacuum in the syringe to the needle. The valve disclosed by Baldwin has a third, vent position in which the needle lumen can be opened to ambient after the specimens are collected and before the needle is removed from the tissue, while at the same time occluding the opening to the syringe. Two problems with these devices are that there is no provision for adequately retaining sufficient quantities of specimens or for transferring the specimens in a controlled manner. The Markham device has the conventional small collection chamber at the of the needle, and the Baldwin device merely contemplates collecting the specimens in the lumen of the needle and expressing them back through the needle under air pressure from the syringe. In fact, the Baldwin patent even acknowledges that in practice some of the accumulated specimens will enter into the vacuum area (within the syringe barrel).

Another reason for the reflux problem is that the proximal end of the needle is typically adjacent the accumulated specimens such that gravity or the pressure differential between the needle and collection chamber can readily force specimens back through the needle. U.S. Pat. No. 5,027,827 to Cody et al. discloses an FNAB device, essentially addressing this issue with the intention of preventing contamination, employing a considerably different arrangement of components. Here, the needle is mounted directly to the syringe barrel and the piston forms a hollow vacuum chamber with a septum at the leading end. When piston is pushed down into the barrel, the septum is punctured by a longitudinal tubular extension, leading to the needle, sticking straight up from the bottom of the barrel. A small opening in the side of the syringe barrel allows the needle to be inserted into the specimens and the piston advanced within the barrel without introducing air into the patient's body. When the piston is advanced far enough so that the septum penetrated by the tubular extension, the needle is evacuated and specimens can be collected into the hollow piston. After the biopsy procedure is completed, this arrangement allows the physician to advance the piston while holding his or her finger over the side opening in the barrel so as to safely blow out any residual biopsy material left in the needle lumen into a biohazard receptacle, while the accumulated specimens remain isolated inside the piston. This arrangement also allows the vacuum in the needle to be achieved and released with minimal loss of the accumulated specimens by simply sliding the piston in and out of engagement with the tubular extension. When the piston is fully retracted into the barrel, the tubular extension extends up above a recessed part of the septum so that when a small amount of specimens is collected, the opening to the pathway of the needle may be spaced from the accumulated specimens. However, reflux is likely when a relatively large amount of specimens is collected, as when a significant portion of the hollow piston was filled. Moreover, the operation of releasing the vacuum in the needle requires the piston to move out of the barrel, which moves the accumulated specimens in the piston proximate the opening in the tubular extension. Consequently, the reflux problem would remain. Another issue with this device is that it is designed for use with a centrifuge, and thus, given the length of the piston, is not well suited for the pick and spread transfer method.

Accordingly, an advanced FNAB device is needed in the art allowing physicians to offer patients a more reliable and higher yield non-surgical biopsy procedure.

### SUMMARY OF THE INVENTION

The present invention provides a needle aspiration biopsy, or FNAB, device, with an anti-reflux head designed specifically to increase the yield of specimens obtained during the biopsy procedure. The anti-reflux head transfers the specimens from the needle to the top of a collection well in a wide-mouth hub. Preferably, the vacuum in the needle is initiated and terminated while the needle is inserted into the tissue. Moreover, the needle can have special side scoops for increasing the amount of specimens collected on each pass of the needle through the specimen sample site, such as a tumor, lesion or other soft tissue region of interest.

In particular, one aspect of the present invention provides a high specimen yield anti-reflux head for a needle aspiration biopsy device including a unique hub. The hub defines a collection wall and mounts a needle with an open pointed tip. A passageway is defined by one or both of the needle and hub which extends from the pointed tip of the needle to a segment extending inside the hub opening in spaced relation to a floor of the collection well.

In one preferred form, the entire passageway is defined by the needle. In this case, the hub has an opening in the floor of the collection well through which a straight segment of the needle shaft extends. The hooked end can take on a modified C-shape configuration so as to follow the contour of the collection well. A segment of the passageway thus follows a sideways or laterally extending path relative to a long axis and opens at a side opening inside the collection well. This lateral segment preferably extends and opens about a lateral axis which is preferably essentially perpendicular to the long axis.

In another preferred form, the passageway is defined in part by the needle and in part by the hub. Specifically, the needle is hollow and straight between its ends and the hub is formed with an internal channel which defines the laterally extending segment. The straight, preferably barbed, proximal end of the needle fits into an opening in the hub in communication with the internal channel. The needle can also be a conventional needle attached to the hub using a standard Luer type locking connection. In either case, the channel can follow a similar C-shaped contour and have the sideways segment and side opening.

In another preferred forms, the collection well has an anti-coagulant surface, preferably being an ACD or EDTA coating, and the needle has a silicon coated lumen and a Teflon coated exterior. The hub has a wide mouth for enhanced access to collected sample inside the collection well. The collection well defined by the hub is relatively large having a volume of at least 100 μL and more preferably a volume of 500 μL to 1 mL or more.

In yet another preferred form, the needle has one or more scoop openings that open to one or more sides of the needle and which are in communication with the passageway. The scoop opening can be of any suitable configuration, such as oblong or round, and preferably extend through the walls at an oblique angle not perpendicular to the long axis of the needle.

In still other preferred forms, the head can be stored before and after use in a sheath stand which defines an elongated cavity containing the needle and has an open end that is mountable to the hub. Preferably, the sheath stand has a wide base for standing the head upright with the needle pointing down and the hub opening upwardly. A cover can be used to seal the collection well so that specimens can be stored in the head after the biopsy procedure.

Another aspect of the invention provides an anti-reflux FNAB device. The FNAB device includes a syringe with a barrel and a sliding piston, a valve for controlling flow to and from the syringe and the anti-reflux head described above. One or more piston locks can be snapped onto the shaft of the piston to hold in place and maintain the vacuum. The FNAB device can also include a sheath stand as described above to conceal the needle as well as to support the device upright. The valve gives the physician or technician precise, one-handed control of the vacuum at the needle so that it can be both initiated and terminated while the needle is within the specimen sample site. This establishes a vacuum suitable to withdraw specimens into the collection well while preventing reflux back into the needle.

In another aspect of the invention the device thus provides for an improved needle aspiration biopsy procedure using the new FNAB device. The procedure or method includes the steps of: creating a vacuum in the syringe; inserting the needle into a specimen sample site; creating a vacuum in the needle; probing the site with the needle; collecting specimens in the hub; releasing the vacuum in the needle; withdrawing the needle from the site; separating the hub from the device; and transferring specimens collected within the hub to an examination site. Preferably, the vacuum is achieved in the syringe by closing the valve and pulling the syringe piston away from the syringe barrel, and the vacuum is achieved in the needle by opening the valve. The vacuum in the needle is resealed simply by reclosing the valve.

The objects and advantages of the present invention will be apparent from the description which follows. The following description merely provides preferred embodiments of the invention. Thus, the claims should be looked to in order to understand the full scope of the invention.

### BRIEF DESCRIPTION OF THE DRAWINGS

FIG. 1 is a perspective view of a preferred embodiment of the FNAB device according to the present invention;

FIG. 2 is a cut-away perspective view of an anti-reflux head thereof;

FIG. 2A is a top view thereof looking into a specimen collection well;

FIG. 3 is a cross-sectional view taken along line 3-3 of FIG. 2A;

FIG. 4 is a perspective view of the FNAB device with a syringe piston pulled from its barrel;

FIG. 5 is another perspective view of the FNAB device with the syringe piston in the barrel and showing a sheath stand for concealing the needle and supporting the device upright;

FIG. 6 is another perspective view of the device shown supported by the sheath stand;

FIG. 7 is a perspective view showing the anti-reflux head containing collected specimens and being covered and inserted into the sheath stand;

FIG. 8 is a perspective view of the covered head in the sheath stand;

FIG. 9 is a side view of an alternate embodiment of the FNAB device having a hook ended needle and a coupler with a separate valve section;

FIG. 10 is an enlarged partial sectional view of the anti-reflux head of the embodiment of FIG. 9;

FIG. 11 is a partial sectional view showing the tip of the needle used in the embodiment of FIG. 9, which is also suitable for use in any of the other described embodiments;

FIG. 12 is a partial perspective view of the tip of the needle showing an oblong side scoop near the tip of the needle;

FIG. 13 is a view similar to FIG. 12 showing a circular side scoop;

FIG. 14 is a partial sectional view showing an alternate sheath stand configuration for the embodiment of FIG. 9, or any other embodiment described herein;

FIG. 15 is a partial sectional view of another alternate embodiment of the FNAB device having a built-in centrifuge vial; and

FIG. 16 is a partial section view of another embodiment of the FNAB device having a conventional needle and Luer type connection of the needle to the hub.

### DETAILED DESCRIPTION OF THE PREFERRED EMBODIMENTS

The present invention provides an FNAB device with an anti-reflux head designed specifically to increase the yield of tissue specimens obtained during the biopsy procedure. The anti-reflux head transfers the specimens from the needle and deposits them down into a collection well in a wide-mouth hub. Preferably, the vacuum in the needle is initiated and terminated while the needle is inserted into the specimen sample site, which could be a tumor, lesion or other soft tissue region of interest. Moreover, the needle can have special side scoops for increasing the specimen yield from each pass of the needle through the site.

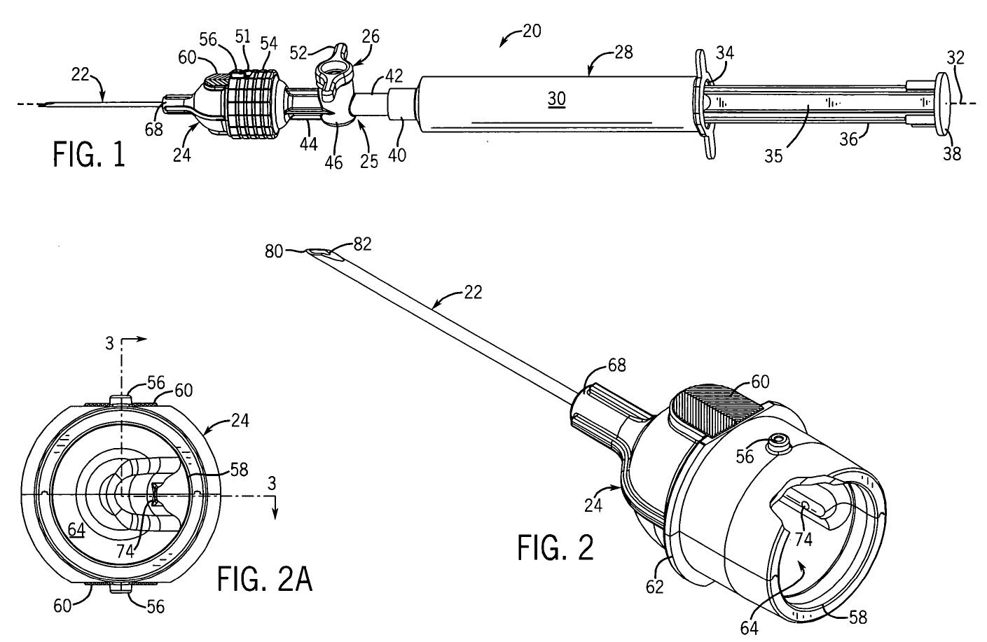

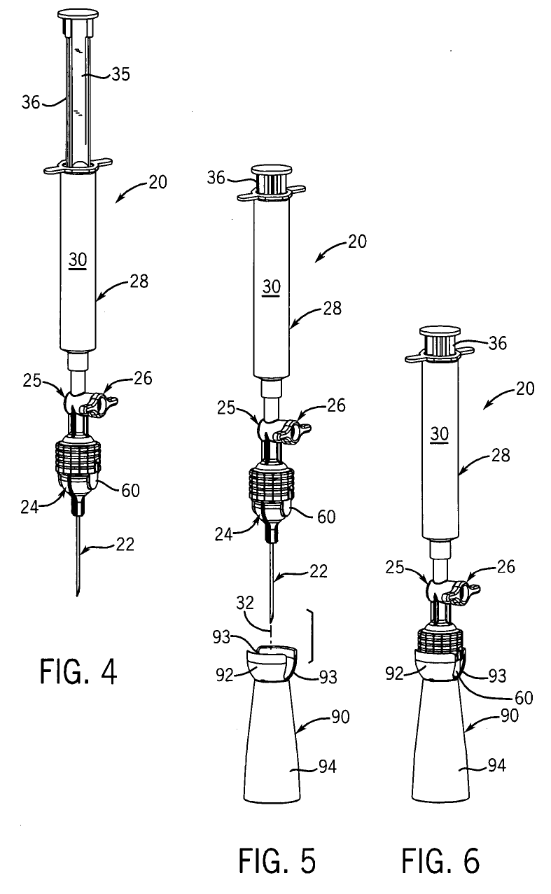

Referring now to FIGS. 1 and 4, the FNAB device 20 generally includes a needle 22 mounted to a hub 24 which is connected by a coupler 25, having a valve 26, to an end of a syringe 28. The syringe 28 is generally of standard construction having a tubular barrel 30, extending along a longitudinal axis 32, with a narrow opening (not shown) at one end and a wide opening 34 at the other end through which a piston 36 can slide inside of the barrel 30. As is conventional, the piston 36 has a head (not shown) that seals against the inner diameter of the barrel 30 to trap gas or liquid within the barrel 30 between the piston head and the narrow end. Preferably, the exposed end of the piston 36 has an enlarged platform 38 for depressing and pulling the piston 36 with respect to the barrel 30. One or more piston locks 35 (one shown in FIGS. 1 and 4) snaps onto the piston 36 between the end of barrel 30 and the platform 38 of the piston 36 to keep the piston in an extended position (pulled out from the barrel 30) to maintain the vacuum in the syringe without holding the piston 36.

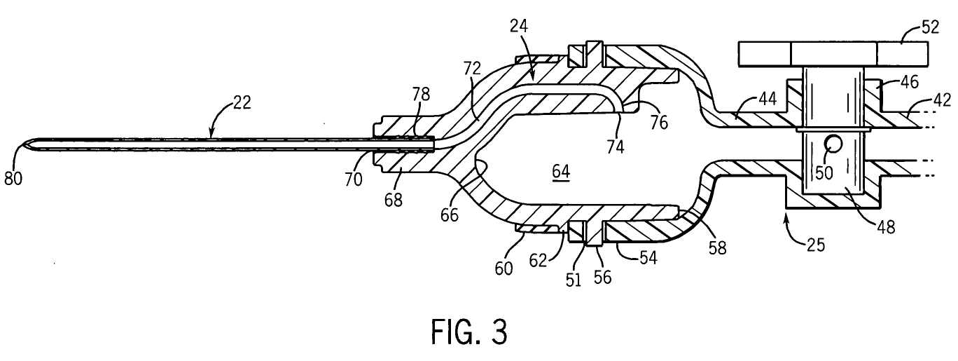

At the narrow end of the syringe 28, a neck 40 receives a tubular longitudinal extension 42 of the coupler 25, which is preferably a suitable plastic, such as a polystyrene or a polycarbonate having good sealing properties. This connection is generally liquid and air tight by the close fit of the mating components. If necessary, this connection can be permanently joined and sealed by a bonding agent or a fusion technique such as ultrasonic welding. Referring to FIG. 3, the extension 42 defines a passageway from the barrel 30 of the syringe 28 to another longitudinal extension 44 of the coupler 25 opposite a lateral cylindrical section 46 housing a cylindrical body 48 of the open-shut valve 26 with an axial opening 50 therethrough. The body 48 is rotated about its lateral axis by a turn lever 52 at the end of the body 48. The valve body 48 and turn lever 52 are preferably formed as one piece of a suitable plastic, such as high or low density polyethylene. The lever 52 can be rotated 90 degrees to align the opening 52 in the body 48 with the passageways of the extensions 42 and 44 of the coupler 25. Rotating the lever 52 90 degrees into the position shown in FIG. 3 again closes off communication between the passageways of extensions 42 and 44. The extension 44 leads to a wide collar 54. The collar 54 has a feature for removably coupling the hub 24. In one preferred form, the collar 54 is formed with a groove 51 opening at the open end of the collar 54 and having axial and lateral runs. The groove 51 receives a boss 56 projecting from the hub 24 in a bayonet type connection in which the boss 56 slides into the axial run of the groove 51 and then the lateral run as the hub 24 is rotated with respect to the collar 54.

Referring to FIGS. 2, 2A and 3, the hub 24 has a wide mouth 58 sized to fit just inside the collar 54. The hub 24 is preferably made of a translucent plastic, or more preferably a transparent plastic, such as transparent polycarbonate, polystyrene, or polypropylene, allowing visual inspection inside the hub 24. The exterior of the hub 24 defines two flat, ribbed grip pads 60 that are 180 degrees apart, allowing the FNAB device 20 to be held with a pencil grip during the biopsy and when disengaging the hub 24 from the coupler 25. A generally annular lip 62 provides a stop feature by abutting the end of the collar 54. The mouth 58 of the hub 24 forms the opening to a collection well 64, having a floor 66, defined by the interior of the hub 24. The hub 24 also defines a narrowed end 68 with an opening 70 from which an internal channel 72 leads to an interior side opening 74 that opens to the collection well 64. The channel 72 follows a gentle C-shaped path from the narrow hub opening 70 and defines a sideways or laterally extending segment 76 adjacent the side opening 74. The side opening 74 opens about, and the lateral segment 76 extends along, an axis essentially perpendicular to the long axis 32 of the device. The channel 72 thus defines a path for specimens to pass to the interior of the hub 24 and into the collection well 64. Importantly, the specimens are deposited down into the collection well 64 from near the top of the well through the side opening 74, which is spaced longitudinally from the floor 66 of the collection well 64. The specimens are not injected into the syringe barrel or up in the direction of the valve. The collection well 64 defines an interior volume of at least 100 μL, and preferably 500 μL to 1 mL (or 1 cc) or more allowing a corresponding volume of specimens to be collected therein. The diameter of the hub mouth is preferably about 1 to 2 centimeters, more preferably of about 1.5 cm. The collection well also has an anti-coagulant coating, such as acid citrate dextrose (ACD) or ethylene diamine tetracetic acid (EDTA), which is designed to prevent the collected sample material from coagulating and attaching to the interior surface of the collection well 64, which would reduce the quantity and compromise the quality of specimens that could be examined.

The narrow hub opening 70 receives a straight proximal end 78 of the needle 22. The proximal end 78 is barbed to resist separation of the needle 22 from the hub 24 during the biopsy procedure. The needle 22 is preferably a fine gauge, such as 18-25 gauge, surgical stainless steel of any suitable length, such as 1 to 8 centimeters. Longer lengths particularly can be adapted for use with radiological or other guidance systems. The needle 22 is hollow from the proximal end 78 to a pointed tip 80 defining an internal, preferably silicon coated, lumen. A flat is formed at the side of the needle 22 near the tip 80 to intersect the opening and form a concave scoop 82 generally oriented axially for increased specimen yield. The open tip 80 and the hollow needle 22 thus combines with the internal passageway 72 of the hub 24 to define a path for specimens to pass into the collection well 64. Also, the exterior surface of the needle 22 is preferably coated with an anti-friction substance, such as Teflon®, to promote good sliding of the needle 22 within the specimen sample site and thereby inhibit the specimens from sticking to the exterior of the needle 22. The needle 22 may also be specially coated with an acoustic media for ultrasound guided FNAB. Further, the shaft of the needle 22 may have graduated markings to show the depth of the needle in the sample tissue during the biopsy procedure.

Referring now to FIGS. 5 and 6, the FNAB device 20 can also include a sheath stand 90 for covering the needle 22 and supporting the hub 24, coupler 25 and syringe 28 in an upright position, as shown in FIG. 6. The sheath stand 90 has a retainer cup 92 that receives the hub 24, with side openings 93 accommodating the grip pads 60, and a hollow base 94 supporting the retainer cup 92. An opening (not shown), generally concentric with the long axis 32, at the bottom of the retainer cup 92 allows the needle 22 to pass into the interior of the base 94. If desired, provisions can be made for the hub 24 to snap into the retainer cup 92 so that the sheath stand 90 stays attached to the hub 24 during transport or when not in the upright position.

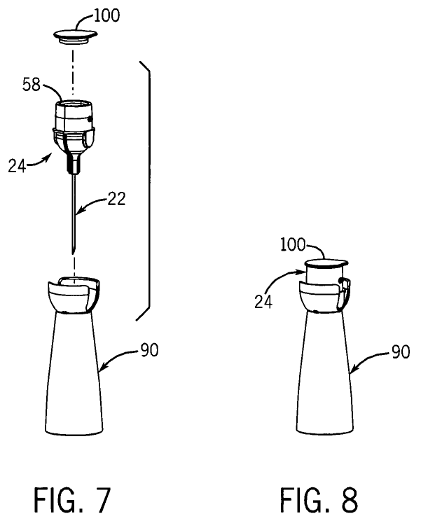

The sheath stand 90 thus allows the FNAB device 20 to be in an upright position before use and following a biopsy procedure. In particular, the sheath stand 90 can be used with the head (hub 24 and needle 22) removed from the coupler 25 and syringe 28, as shown in FIGS. 7 and 8. For example, following a biopsy procedure, the head can be removed and a lid 100 can be screwed or snapped over the mouth 58 of the hub 24 to enclose the collection well 64. Thus, the head and sheath stand 90 can be used to store the collected sample material in a safe and secure manner. In this way, the used needle 22 is concealed and guarded from accidental contact, and the collected specimens are kept contained in the collection well 64 of the hub 24 with the lid 100 preventing accidental spillage or loss of specimens.

The above described device can thus be used advantageously in a needle aspiration biopsy procedure that improves the yield of specimens overall and the quantity of specimens collected on each pass of the needle 22 through the specimen sample site. The device can be used in human and veterinary medicine on living patients as well as in various research applications. Generally, the biopsy procedure achieves improved yield because of the unique features of the FNAB device 20, primarily the way specimens are delivered into the collection well 64 and by achieving, sustaining and releasing a vacuum in the sample collection path while the needle 22 is inserted into the specimen sample site. More specifically, the method includes: creating a vacuum in the syringe; inserting the needle into the sample area; creating a vacuum in the needle; probing the specimen sample site with the needle; collecting specimens in the hub; releasing the vacuum in the needle; withdrawing the needle from the site; separating the hub from the device; and transferring specimens collected inside the hub to an examination site.

Still more specifically, in one preferred method of the inventive biopsy procedure, the above described device is held by the user (physician, nurse, veterinarian, researcher, etc.) and the turn lever 52 is rotated until the valve 26 is closed. The syringe piston 36 is then pulled away from the barrel 30 to create a vacuum within the barrel 30 and the piston lock(s) are snapped onto the shaft of the piston 36. After sufficiently cleaning, sterilizing and otherwise preparing the specimen sample site, the needle 22 is inserted into the site. Then, the turn lever 52 is rotated ¼ turn to open the valve 26. This transfers the air in the needle 22 up into the syringe barrel 30 and effectively creates a vacuum in the needle 22. The FNAB device 20 is then moved up and down (or back and forth) so that the needle 22 repeatedly probes and aspirates the specimen sample site without the tip of the needle coming out of the site and leaking the vacuum. Specimens pass into the tip and side scoop 82 openings of the needle 22 under vacuum as the device is moved, and specimens are drawn into the channel 72 of the hub 24 where they will pass through the side opening 74 and from there fall down into the collection well 64. Specimens will continue to pile up in the collection well 64, and when sufficient specimens for analysis have been collected (which can be checked visually through the transparent hub) and before the needle 22 is removed from the site, the turn lever 52 is again turned ¼ turn so that the valve 26 is once again closed. This will disrupt the vacuum in the needle 22. The collection well 64 is large enough to accumulate a diagnostically sufficient quantity of specimens without reaching the height of the side opening 74. The collected specimens are thus spaced from the path back to the needle 22. This, and the fact that the specimens would have to travel sideways relative to the needle 22 (thus without the assistance of gravity) to pass through the lateral segment 76, prevents loss of specimen into the syringe barrel and by reflux back into and through the needle.

Following the procedure, the anti-reflux head can be separated from the rest of the FNAB device 20 by uncoupling the hub 24 from the collar 54 by holding grips 60. The wide mouth 58 of the hub 24 allows access to the collected sample material with conventional gripping instruments, such as forceps or micropipette tips, which can be used to transfer a controlled amount of specimens into a test tube or vial or onto a microscope slide or other examination area. When the FNAB device 20 includes the sheath stand 90, the head can be placed needle first into the sheath stand 90 so that the hub 24 rests securely in the retainer cup 92. If some or all of the collected specimens are to be stored rather than processed immediately, the lid 100 can be secured onto the mouth 58 of the hub 24.

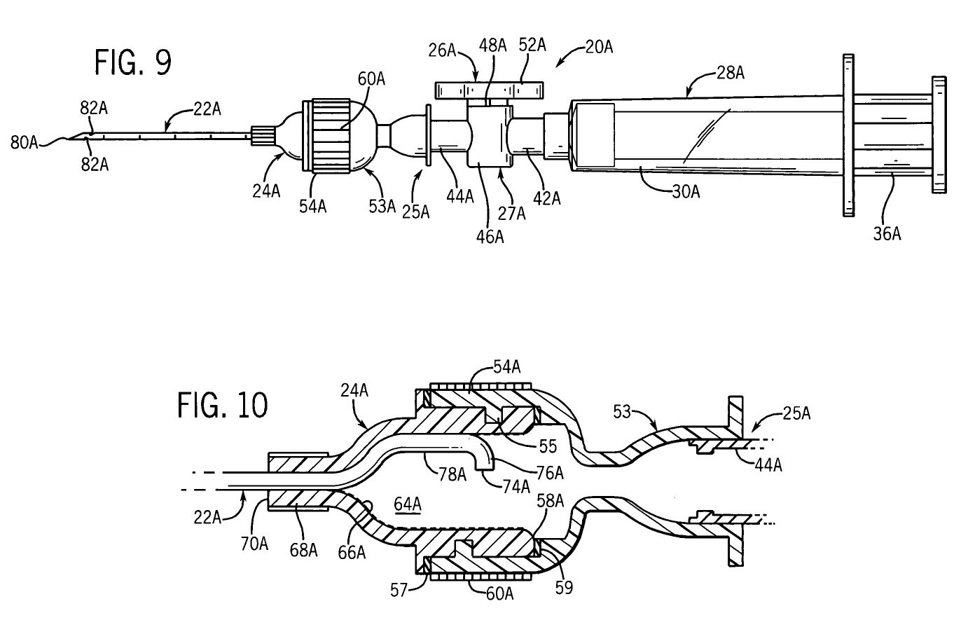

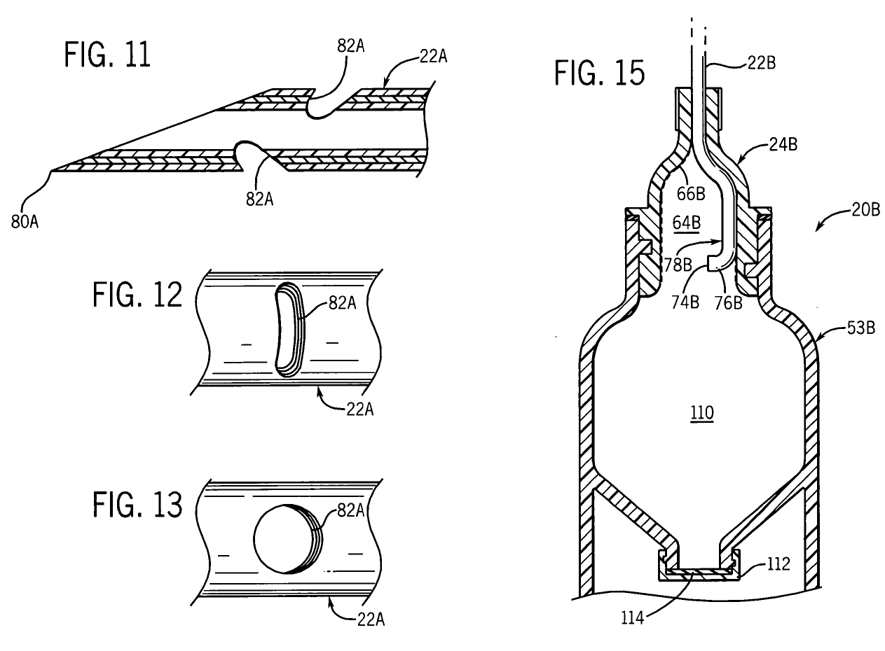

FIGS. 9-14 illustrate an alternate embodiment of the device in which the anti-reflux head has a different needle and hub configuration to accomplish the functional one-way valve arrangement through which the specimens are deposited into the collection well. In this embodiment like components will be referred to herein using like reference numerals albeit with the suffix “A” attached.

Here, the FNAB device 20A includes a needle 22A mounted to a hub 24A which is connected to a coupler 25A attached to a valve 26A and mounted at the end of a syringe 28A. Although shown in the drawings of slightly different configuration, the syringe 28A in this embodiment can be identical to that previously described, including a standard type barrel 30A and sliding piston 36A. The narrow end of the syringe 28A receives a tubular extension 42A of one part 27A of the valve 26A, which has another longitudinal extension 44A and a lateral T-section 46A housing a valve body section 48A with an axial opening 50A. As above, the valve body 48A is rotated about its lateral axis by a turn lever 52A to open and close the valve 26A. Here, the extension 44A is connected to coupler part 53A that includes a wide collar 54A having ridges about its periphery for gripping. In this embodiment, the collar 54A has male threads 55 that engage the exterior of the hub 24A, and two gaskets or seals 57 and 59 can be disposed between the hub 24A and the collar 54A to prevent leakage.

Like before, the hub 24A is large and has a wide mouth 58A sized to fit just inside the collar 54A and forming the opening to a collection well 64A, having a floor 66A. The hub 24A also defines a narrowed end 68A with an opening 70A that opens at the bottom of the collection well 64A. As mentioned, the principal distinguishing feature of this embodiment is the arrangement of the needle and hub defining the pathway for the specimens into the collection well 64A. Rather than an internal passageway in the hub 24A, the needle 22A is formed with a unique hooked or C-shaped proximal end 78A, some of which preferably follows the contour of the collection well 64A to better stabilize the needle 22A. The proximal end 78A defines a lateral segment 76A ending in a side opening 74A. The proximal end 78A is at the end of a straight shaft of the needle 22A that fits through the narrow hub opening 70A. As before, the needle 22A is hollow from the proximal end 78A to a pointed tip 80A, however, here two side scoops 82A are located a short distance up the shaft from the tip 80A. As shown in FIG. 12, the scoops 82A are preferably oblong, however, they may be of any other suitable configuration, such as circular, as shown in FIG. 13. Scoops 82A of these configuration provide on the order of three times the cutting surface of the needle tip opening and thereby increase the yield of specimens significantly on each pass.

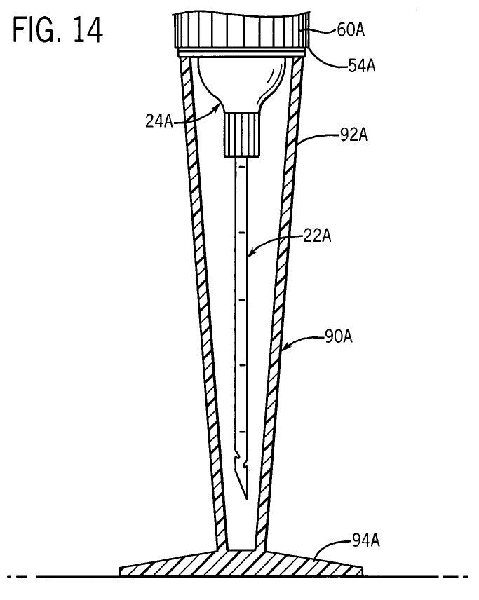

Thus, in this embodiment the needle 22A defines the entire passageway for the specimens to reach the collection well 64A. This alternate configuration retains the same type of anti-reflux one way flow by depositing the specimens through the side opening 74A, which is spaced from the well floor 66 near the mouth 58A. And, as before the collection well 64A has an anti-coagulant coating and the outside of the needle has an anti-friction coating. As shown in FIG. 14, this embodiment of the device can also include a sheath stand 90A for covering the needle 22A and supporting the FNAB device 20A upright. Here, the sheath stand 90A has a long, narrow retainer cup 92A that receives the hub 24A and the needle 22A and is supported by a wide flat base 94A.

FIG. 15 shows the head of another alternate embodiment of the inventive FNAB device 20B, which is similar to the last described embodiment, having a needle 22B with a hooked proximal end 78B and a separate hub 24B defining a collection well 64B with a floor 66B spaced from a side opening 74B of a lateral segment 76B. In this embodiment, a separate collection vial 53B can be assembled with the hub 24B. The collection vial 53B defines an interior volume 110 into which the specimens can be transferred and enclosed by a cap 112 with a seal 114. The vial 53B can be used in standard laboratory centrifuges.

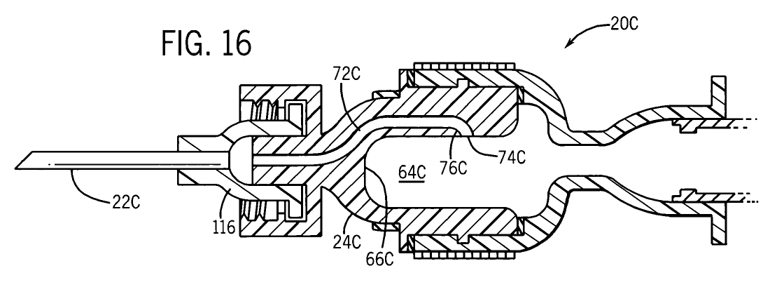

FIG. 16 shows the head of yet another alternate embodiment of the inventive FNAB device 20C, which is similar to the first embodiment, in which a straight needle 22C and is connected to a separate hub 24C having a C-shaped channel 72C with a lateral segment 76C and side opening 74C opening to a collection well 64C with a floor 66C spaced from the side opening 74C. In this embodiment, the needle 22C is a conventional, straight hypodermic needle with a standard female Luer connection end 116 which removably snaps onto the narrow neck of the hub 24C configured in a standard male Luer connection. The Luer connection can be a locking or slip type connection, the locking type being preferred. This allows the FNAB device 20C to accept standard needles with standard connections. Since no special needle is required, the device can be made and used even more cost effectively and operated even more intuitively. The anti-reflux and high yield characteristics of the FNAB device are retained by the configuration of the hub 24C.

The inventors of the present invention have conducted empirical studies establishing the efficacy of the device and method described herein. One comparative study was conducted using the second described embodiment of the FNAB device shown in FIGS. 9 and 10. The study included taking five trial biopsies using this FNAB device and five trial biopsies using a conventional syringe and hypodermic needle. Each set of five trial biopsies were performed four times so that there were 20 samples for the new FNAB device and the conventional device. All of the biopsies were taken from cattle liver and using the same number of probes to a generally uniform depth. The trials using the conventional device yielded between about 17 and 30 μL of tissue, while those using the FNAB of the present invention yielded between about 70 and 100 μL of tissue. Cell blocks were formed from the yield from each of the ten trial biopsies and cytology smears were conducted for each trial. The cytology smears were observed without any type of image enhancement, yet significantly improved cellularity and tissue density was apparent for each trial using the new FNAB device when compared to the trials from the conventional device. It was observed that the smears from specimens obtained with the new FNAB device had cells covering at least 20-50% and in some cases more than 50% of the smear field, compared to those from the conventional device which covered less than 20% of the field with cells and in some cases had only a few cells scattered across the field. The study thus supports the position that the new FNAB device provides consistently higher yields of sample tissue, on the order of 300 to 500 percent, and affords physicians a consistently improved cytology diagnosis procedure.

Illustrative embodiments of the present invention have been described above in detail. However, the invention should not be limited to the described embodiments since many modifications and variations to the preferred embodiments, apparent to those skilled in the art, will be within the spirit and scope of the invention. Therefore, to ascertain the full scope of the invention, the following claims should be referenced.

1. A high specimen yield anti-reflux head for a needle aspiration biopsy device, comprising:
a hub defining a specimen collection well and mounting a needle having a shaft with an open pointed tip; and
a sample passageway extending from the pointed tip of the needle to a segment inside the hub opening in spaced relation to a floor of the collection well.

2. The device of claim 1, wherein the needle defines the entire passageway extending from the pointed tip to a contoured proximal end.

3. The device of claim 2, wherein the hub defines an opening in the floor of the collection well through which the needle shaft extends.

4. The device of claim 2, wherein the proximal end of the needle includes a segment that extends along and opens about a lateral axis at an angle to a longitudinal axis of the needle.

5. The device of claim 4, wherein the lateral and longitudinal axes are essentially perpendicular.

6. The device of claim 3, wherein the proximal end of the needle in part follows the contour of the collection well.

7. The device of claim 1, wherein the passageway is defined in part by the needle and in part by an internal channel in the hub.

8. The device of claim 7, wherein the needle has a straight proximal end disposed at an opening in the hub defining an end of the channel.

9. The device of claim 8, wherein the proximal end of the needle has raised barbs.

10. The device of claim 8, wherein the channel includes a lateral segment that extends along and opens about a lateral axis at an angle to a longitudinal axis of the needle.

11. The device of claim 10, wherein the lateral and longitudinal axes are essentially perpendicular.

12. The device of claim 1, wherein the collection well has an anti-coagulant surface.

13. The device of claim 12, wherein the anti-coagulant surface is a coating of ACD or EDTA.

14. The device of claim 1, wherein the needle has an anti-friction surface.

15. The device of claim 14, wherein the anti-friction surface is a Teflon coating.

16. The device of claim 1, wherein the hub includes an outer grip.

17. The device of claim 15, wherein the hub has an open mouth allowing access to the collection well.

18. The device of claim 17, further including a lid securable to the hub to cover the mouth.

19. The device of claim 1, wherein the collection well has a volume of at least 100 μL.

20. The device of claim 1, further including a sheath stand defining an elongated cavity for containing the needle and having an open end mountable to the hub.

21. The device of claim 1, wherein the needle defines a scoop opening at a side of the needle in communication with the passageway.

22. A high specimen yielding anti-reflux needle aspiration biopsy device, comprising:
a syringe including a barrel and a piston slidable within the barrel;
a valve for controlling an opening in the syringe barrel;
a hub linked to the valve and defining a specimen collection well; and
a needle mounted to the hub having a shaft with an open pointed tip;
wherein one or more of the hub and needle define a passageway extending from the needle tip to inside the hub opening in spaced relation to a floor of the collection well.

23. The device of claim 22, further including a coupler containing the valve and connecting the hub to the syringe.

24. The device of claim 22, wherein the needle defines the entire passageway extending from the pointed tip to a contoured proximal end.

25. The device of claim 22, wherein the passageway is defined in part by the needle and in part by an internal channel in the hub.

26. The device of claim 25, wherein the needle has a straight proximal end disposed at an opening in the hub defining an end of the channel.

27. The device of claim 22, wherein the collection well has an anti-coagulant surface and the needle has an anti-friction exterior surface.

28. The device of claim 22, further including a sheath stand defining an elongated cavity for containing the needle and having an open end mountable to the hub.

29. The device of claim 22, further including a piston lock mounted to the syringe so as to fix the position of the piston relative to the barrel.

20. The device of claim 22, wherein the needle defines a scoop opening at a side of the needle in communication with the passageway.

31. A method of needle aspiration biopsy using a device as recited in claim 22, comprising the steps of:
creating a vacuum in the syringe;
inserting the needle into a specimen sample site;
communicating the vacuum to the needle;
probing the specimen sample site with the needle to collect specimens in the collection well of the hub;
releasing the vacuum in the needle;
withdrawing the needle from the specimen sample site;
separating the hub from the device; and
transferring specimens collected in the hub to an examination site.

32. The method of claim 31, wherein the step of creating a vacuum in the syringe includes closing the valve and pulling the syringe piston away from the syringe barrel.

33. The method of claim 32, wherein the vacuum is communicated to the needle by opening the valve.

34. The method of claim 33, wherein the step of releasing the vacuum in the needle includes reclosing the valve.

35. A high specimen yielding anti-reflux needle aspiration biopsy device, comprising:
a syringe including a barrel and a piston slidable within the barrel;
a valve for controlling an opening in the syringe barrel;
a hub linked to the valve and defining a specimen collection well having a volume of more than 500 micro liters; and
a needle mounted to the hub having a shaft with an open pointed tip;
wherein one or more of the hub and needle define a passageway extending from the needle tip to inside the collection well.

37. A high specimen yielding anti-reflux needle aspiration biopsy device, comprising:
a syringe including a barrel and a piston slidable within the barrel;
a valve for controlling an opening in the syringe barrel; and
a hub linked to the valve and defining a specimen collection well, wherein the hub defines an internal passageway for putting the collection well in communication with a lumen of a needle.

38. The device of claim 37, wherein the collection well has an interior volume of at least 100 micro liters.

39. The device of claim 37, wherein the internal passageway opens to an interior of the collection well through an opening spaced from a floor of the collection well.
